(19) United States
(12) Patent Application Publication
(10) Pub. No.: US 2015/0251024 A1
(43) Pub. Date: Sep. 10, 2015

(54) SPATIAL CONFIGURATION DETERMINATION APPARATUS

(71) Applicant: KONINKLIJKE PHILIPS N.V., Eindhoven (NL)

(72) Inventors: Harm Jan Willem Belt, Weert (NL); Steven Antonie Willem Fokkenrood, 's-Hertogenbosch (NL); Szabolcs Deladi, Veldhoven (NL)

(73) Assignee: KONINKLIJKE PHILIPS N.V., EINDHOVEN (NL)

(21) Appl. No.: 14/432,777

(22) PCT Filed: Oct. 15, 2013
(86) PCT No.: PCT/IB2013/059366

§ 371 Date: Apr. 1, 2015

Related U.S. Application Data
(60) Provisional application No. 61/717,153, filed on Oct. 23, 2012.

(51) Int. Cl. A61N 7/00; A61B 8/12; A61B 8/08

(52) U.S. Cl. CPC A61N 7/00; A61B 8/486; A61B 8/12; A61B 8/5223

(57) ABSTRACT

The invention relates to a spatial configuration determination apparatus for determining a spatial configuration in the surrounding of an ultrasound device, in particular, for determining the orientation of the ultrasound device and of an object in the surrounding of the ultrasound device with respect to each other. The ultrasound device is preferentially arranged at a tip (5) of a cardiac ablation catheter (4) such that, for instance, the orientation of the tip (5) relative to cardiac tissue can be determined. Motion data and/or distance data are determined in different directions from ultrasound data acquired by the ultrasound device, wherein the determined motion data and/or distance data are used for determining the spatial configuration. This allows determining, for instance, the orientation of a tip (5) of an ablation catheter (4) relative to cardiac tissue, without necessarily requiring further orientation determination means.

### FIELD OF THE INVENTION

The invention relates to a spatial configuration determination apparatus, a spatial configuration determination method and a spatial configuration determination computer program for determining a spatial configuration in the surrounding of an ultrasound device. The invention relates further to an introduction apparatus comprising the spatial configuration determination apparatus.

### BACKGROUND OF THE INVENTION

In cardiac ablation procedures an ablation catheter is introduced into a heart of a living being, wherein the tip of the ablation catheter comprises, for instance, an ablation electrode for applying radio frequency energy to cardiac tissue to be ablated. Moreover, the tip of the ablation catheter can comprise an ultrasound transducer for ultrasonically monitoring the ablation procedure. For determining the orientation and position of the tip of the ablation catheter within the heart the tip is generally electromagnetically tracked, wherein an electromagnetic sensor placed in the tip of the ablation catheter senses varying magnetic fields generated by an external field generator and wherein the orientation and the position of the tip of the ablation catheter is calculated based on the sensed varying magnetic fields.

A disadvantage of using such an electromagnetic technique for determining the orientation and position of the tip of the ablation catheter within the heart is the need to integrate three-dimensional sensors in the already restricted space inside the tip of the ablation catheter and the need to place an additional external magnetic field generator in the laboratory in which the ablation procedure is performed.

### SUMMARY OF THE INVENTION

It is an object of the present invention to provide a spatial configuration determination apparatus, a spatial configuration determination method and a spatial configuration determination computer program for determining a spatial configuration in the surrounding of an ultrasound device, which allow for a determination of a spatial configuration in the surrounding of the ultrasound device, without requiring much space.

In a first aspect of the present invention a spatial configuration determination apparatus for determining a spatial configuration in the surrounding of an ultrasound device is presented, wherein the spatial configuration determination apparatus is adapted to determine the spatial configuration based on acquired first and second ultrasound data, which have been acquired by the ultrasound device in first and second acquisition directions, respectively, wherein the first and second acquisition directions are different and wherein the spatial configuration determination apparatus comprises:

an ultrasound data processing unit for processing the acquired first and second ultrasound data for determining motion data representing motion of an object in the surrounding of the ultrasound device and/or distance data representing a distance between the object and the ultrasound device in the first and second acquisition directions,

a spatial configuration determination unit for determining the spatial configuration in the surrounding of the ultrasound device based on the motion data and/or the distance data determined for the different acquisition directions.

Since the spatial configuration determination unit determines the spatial configuration in the surrounding of the ultrasound device based on the motion data and/or the distance data that have been determined from the acquired first and second ultrasound data for the different acquisition directions, the spatial configuration can be determined without necessarily requiring additional means, thereby allowing the spatial configuration determination apparatus to determine the spatial configuration without requiring much space. For instance, ultrasound data provided by ultrasound transducers arranged at a tip of an introduction element like an ablation catheter can be used for determining the spatial configuration in the surrounding of the tip, wherein the tip of the ablation catheter can still comprise relative small dimensions. Particularly the orientation of the tip of an ablation catheter and of a heart wall with respect to each other, i.e. the orientation of the tip of the ablation catheter relative to the heart wall, can be determined as the spatial configuration just based on the ultrasound data provided by the ultrasound device arranged at the tip of the ablation catheter. The ultrasound transducers at the tip of the ablation catheter may therefore be used for at least two different purposes, ultrasonically visualizing cardiac tissue to be ablated, in order to monitor an ablation procedure, for example, as disclosed in WO 2010/082146 A1, and determining the orientation of the tip of the ablation catheter relative to the heart wall.

The spatial configuration determination unit is preferentially adapted to determine the orientation of the ultrasound device and an object in the surrounding of the ultrasound device with respect to each other as the spatial configuration. For instance, the object can be a wall of, for instance, a heart of a living being, wherein the spatial configuration determination unit can be adapted to determine the orientation of the ultrasound device and the wall with respect to each other, i.e. the orientation of the ultrasound device relative to the wall, as the spatial configuration.

The spatial configuration determination unit can also be adapted to determine the position of the ultrasound device and an object like a heart wall in the surrounding of the ultrasound device with respect to each other as the spatial configuration.

The acquisition directions are directions with respect to the ultrasound device, i.e. if the orientation and/or the position of the ultrasound device is modified, also the acquisition directions are modified. Thus, the ultrasound data acquired in different acquisition directions within the heart depend on the position and/or orientation of the ultrasound device such that the ultrasound data can be used for determining, for instance, the position and/or orientation of the ultrasound device and, thus, of a tip of an interventional instrument like a catheter, if the ultrasound device is attached to the tip.

It is preferred that the spatial configuration determination unit is adapted to determine the orientation and/or the position of the ultrasound device and the object in the surrounding of the ultrasound device with respect to each other as the spatial configuration. The ultrasound data processing unit can be adapted to determine motion data being indicative of the magnitudes and/or the directions of the motion between the ultrasound device and the object in the surrounding of the ultrasound device in the first acquisition direction from the first ultrasound data and of the motion between the ultrasound device and the object in the surrounding of the ultrasound device in the second acquisition direction from the second ultrasound data. The motion in the surrounding of the ultrasound device in the different acquisition directions can strongly depend on the spatial configuration of one or several objects in the surrounding of the ultrasound device, in particular, on the position and/or orientation of the ultrasound device with respect to the one or several objects in the surrounding of the ultrasound device. The motion determined in the different acquisition directions can therefore be used for determining the spatial configuration with high accuracy.

The acquired first ultrasound data preferentially form a first M-mode image and the acquired second ultrasound data preferentially form a second M-mode image. For determining the motion data the ultrasound data processing unit is preferentially adapted to determine first sub-M-mode images from the first M-mode image and second sub-M-mode images from the second M-mode image and to apply a motion determination algorithm to the first sub-M-mode images for determining first motion data being indicative of the motion in the first acquisition direction and to apply the motion determination algorithm to the second sub-M-mode images for determining second motion data being indicative of the motion in the second acquisition direction. In particular, for each acquisition direction two sub-M-mode images are determined and input into the motion determination algorithm for determining the motion in the first and second acquisition directions, respectively. The motion determination algorithm can be, for instance, an optical flow algorithm, a correlation-based algorithm, et cetera.

The ultrasound data processing unit is preferentially adapted to determine the distance data such that the distance data are indicative of a distance between the ultrasound device and an object in the surrounding of the ultrasound device in the first acquisition direction from the first ultrasound data and such that the distance data are indicative of a distance between the ultrasound device and the object in the surrounding of the ultrasound device in the second acquisition direction from the second ultrasound data. In particular, the ultrasound data processing unit can be adapted to determine the distance data by thresholding the first and second ultrasound data, wherein, if an ultrasound value is larger than a predefined threshold, it can be assumed that the object is located at the corresponding distance to the ultrasound device. Since the distance between the ultrasound device and an object in the surrounding of the ultrasound device in the first and second acquisition directions can define the spatial configuration in the surrounding of the ultrasound device, by determining these distances based on the ultrasound data acquired in different acquisition directions the spatial configuration in the surrounding of the ultrasound device can be determined with high accuracy.

The spatial configuration determination unit is preferentially adapted to use a statistical classifier for determining the spatial configuration in the surrounding of the ultrasound device based on the motion data and/or the distance data in the different acquisition directions. The statistical classifier is preferentially adapted to determine which spatial configuration from a set of predefined spatial configurations corresponds most likely to the motion data and/or the distance data in the different acquisition directions, wherein the most likely spatial configuration from the set of predefined spatial configurations is determined as the spatial configuration. The set of predefined spatial configurations can include, for instance, at least one of the group of a spatial configuration, in which the ultrasound device is buried in an object; a spatial configuration, in which the ultrasound device is located within the apex of a heart; a spatial configuration, in which the ultrasound device is located in a trabeculated structure of the heart; predefined orientations of the ultrasound device and an object in the surrounding of the ultrasound device with respect to each other; predefined positions of the ultrasound device and an object in the surrounding of the ultrasound device with respect to each other. This means that the set of predefined spatial configurations can include, for instance, a spatial configuration, in which the ultrasound device is buried in an object, and/or a spatial configuration, in which the ultrasound device is located within the apex of a heart, and/or a spatial configuration, in which the ultrasound device is located in a trabeculated structure of the heart, and/or predefined orientations of the ultrasound device and an object in the surrounding of the ultrasound device with respect to each other and/or predefined positions of the ultrasound device and an object in the surrounding of the ultrasound device with respect to each other. Thus, the spatial configuration determination apparatus cannot only be adapted to determine, for instance, the orientation and/or the position of the ultrasound device with respect to an object in the surrounding of the ultrasound device, but the spatial configuration determination apparatus can also be adapted to recognize special cases like a situation, in which the ultrasound device is buried into tissue, or a situation, in which the ultrasound device reaches into the apex or into a trabeculated structure of the heart.

The ultrasound data are preferentially provided as RF-lines or A-lines. For instance, several A-lines forming an M-mode image can be acquired in the first acquisition direction and several A-lines forming a further M-mode image can be acquired in the second acquisition direction, in order to provide temporally dependent first and second ultrasound data. For determining the motion in the first acquisition direction different A-lines acquired in the first acquisition direction at different points in time can be compared with respect to each other. Correspondingly, also the A-lines acquired in the second acquisition direction can be compared for determining the motion in the second acquisition direction.

The spatial configuration determination apparatus can be adapted to determine the spatial configuration based on more than two ultrasound data. For instance, ultrasound data can be provided, which have been acquired in more than two acquisition directions, wherein for the different acquisition directions different motion data and/or distance data in the surrounding of the ultrasound device can be determined by processing the corresponding ultrasound data acquired in the different acquisition directions and wherein the spatial configuration in the surrounding of the ultrasound device can be determined based on the motion data and/or the distance data determined in the different acquisition directions.

In a further aspect of the present invention an introduction apparatus for introducing an introduction element into an object is presented, wherein the introduction apparatus comprises:

the introduction element to be introduced into the object,

an ultrasound device for acquiring first and second ultrasound data in different acquisition directions, wherein the ultrasound device is arranged at the introduction element,

a spatial configuration determination apparatus as defined in claim 1 for determining the spatial configuration around the ultrasound device within the object based on the first and second ultrasound data.

In a preferred embodiment the ultrasound device is arranged at a tip of the introduction element and comprises a frontal transducer for acquiring the first ultrasound data in a frontal direction with respect to the tip of the introduction element and at least one lateral transducer for acquiring the second ultrasound data in a lateral direction with respect to the introduction element. The frontal transducer can also be regarded as being an axial transducer, because the introduction element is preferentially a longish introduction element defining an axial direction and because the frontal transducer is preferentially adapted to acquire the first ultrasound data in the axial direction. Moreover, the tip of the introduction element can be substantially circular in cross section such that lateral radial acquired directions, wherein in this case the at least one lateral transducer can be regarded as being a radial transducer.

The ultrasound device preferentially comprises at least three lateral transducers for acquiring the second ultrasound data and for acquiring third and fourth ultrasound data, wherein the ultrasound device is adapted such that the first to fourth ultrasound data are all acquired in different acquisition directions, wherein the spatial configuration determination apparatus is adapted to determine the spatial configuration around the ultrasound device based on the first to fourth ultrasound data. This configuration of transducers allows for a further improved determination of the spatial configuration in the surrounding of the ultrasound device, wherein still a relatively small amount of ultrasound transducers is present, which allows providing the introduction element with the ultrasound device with relatively small dimensions.

In a further aspect of the present invention a spatial configuration determination method for determining a spatial configuration in the surrounding of an ultrasound device is presented, wherein the spatial configuration determination method is adapted to determine the spatial configuration based on acquired first and second ultrasound data, which have been acquired by the ultrasound device in first and second acquisition directions, respectively, wherein the first and second acquisition directions are different and wherein the spatial configuration determination method comprises:

processing the acquired first and second ultrasound data for determining motion data representing motion of an object in the surrounding of the ultrasound device and/or distance data representing a distance between the object and the ultrasound device in the first and second acquisition directions by an ultrasound data processing unit,

determining the spatial configuration in the surrounding of the ultrasound device based on the motion data and/or the distance data, which have been determined in the different acquisition directions, by a spatial configuration determination unit.

In a further aspect of the present invention a spatial configuration determination computer program for determining a spatial configuration in the surrounding of an ultrasound device is presented, wherein the computer program comprises program code means for causing a spatial configuration determination apparatus as defined in claim 1 to carry out the steps of the spatial configuration determination apparatus as defined in claim 13, when the computer program is run on a computer controlling the spatial configuration determination apparatus.

It shall be understood that the spatial configuration determination apparatus of claim 1, the introduction apparatus of claim 10, the spatial configuration determination method of claim 13, and the spatial configuration determination computer program of claim 14 have similar and/or identical preferred embodiments, in particular, as defined in the dependent claims.

It shall be understood that a preferred embodiment of the invention can also be any combination of the dependent claims with the respective independent claim.

These and other aspects of the invention will be apparent from and elucidated with reference to the embodiments described hereinafter.

### BRIEF DESCRIPTION OF THE DRAWINGS

In the drawings:

FIG. 1 shows schematically and exemplarily an embodiment of an ablation apparatus for ablating cardiac tissue,

FIG. 2 shows schematically and exemplarily a tip of an ablation catheter of the ablation apparatus,

FIG. 3 shows exemplarily an RF-line and an A-line,

FIG. 4 shows schematically and exemplarily the tip of the ablation catheter contacting a heart wall in a certain orientation,

FIG. 5 shows schematically and exemplarily the tip of the ablation catheter in the left atrium of the heart in another orientation,

FIG. 6 shows schematically and exemplarily the tip of the catheter in the orientation shown in FIG. 5 in more detail in the systole,

FIG. 7 shows schematically and exemplarily the tip of the catheter in the orientation shown in FIG. 5 in more detail in the diastole,

FIG. 8 shows exemplarily ultrasound data measured in different acquisition directions and at different angular orientations of the tip of the ablation catheter relative to the heart wall,

FIGS. 9 and 10 shows exemplarily mobility values for different acquisition directions and different angular orientations of the tip of the ablation catheter relative to the heart wall,

FIG. 11 shows a diagram exemplarily illustrating a procedure for determining the angular orientation of the tip of the ablation catheter relative to the heart wall from acquired ultrasound data,

FIG. 12 shows a diagram to be shown on a display of the ablation apparatus for indicating the determined angular orientation of the tip of the ablation catheter relative to the heart wall,

FIG. 13 shows a diagram exemplarily illustrating a procedure for training a statistical classifier that may be used for determining the angular orientation of the tip of the ablation catheter relative to the heart wall, and

FIG. 14 shows a flowchart exemplarily illustrating an embodiment of a spatial configuration determination method for determining a spatial configuration in the surrounding of an ultrasound device.

### DETAILED DESCRIPTION OF EMBODIMENTS

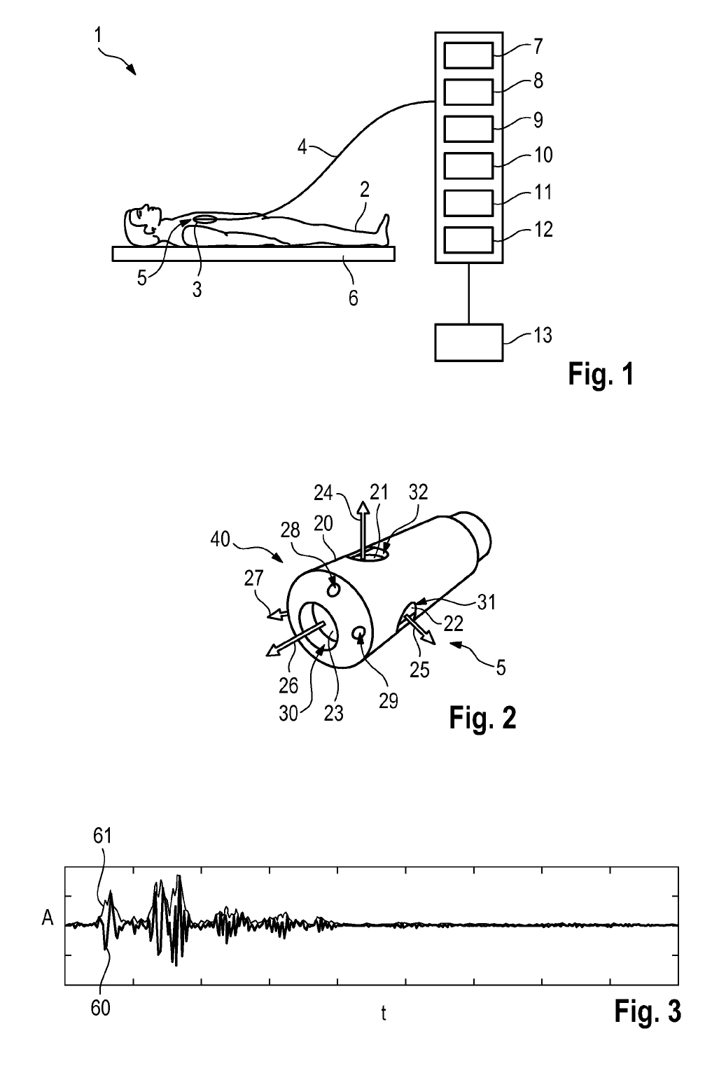

FIG. 1 shows schematically and exemplarily an introduction apparatus for introducing an introduction element into an object. In this embodiment the introduction apparatus is an ablation apparatus 1 for performing a cardiac ablation procedure, wherein the ablation apparatus 1 comprises an ablation catheter 4 being the introduction element for being introduced into a heart 3 of a person 2 lying on a support means 6 like a table. The tip 5 of the ablation catheter 4 is schematically and exemplarily shown in more detail in FIG. 2.

The tip 5 of the ablation catheter 4 comprises an ultrasound device 40 for acquiring ultrasound data in different acquisition directions. In this embodiment the ultrasound device 40 comprises a frontal transducer 23 for acquiring first ultrasound data in a first acquisition direction 26 being a frontal direction with respect to the tip 5 of the ablation catheter 4 and three lateral transducers, of which only two transducers 21, 22 are visible in FIG. 2, for acquiring second, third and fourth ultrasound data in second, third and fourth acquisition directions 24, 25, 27 being lateral directions.

The frontal transducer 23 can be regarded as being an axial transducer acquiring the first ultrasound data in the axial direction 26 being the first acquisition direction. Moreover, in this embodiment the tip 5 of the ablation catheter 4 is substantially circular in cross section such that the lateral directions 24, 25, 27 can be regarded as being radial directions and the respective lateral transducers can be regarded as being radial transducers.

The tip 5 of the ablation catheter 4 further comprises an ablation electrode 20 comprising an axial opening 30 and lateral openings 31, 32, through which the ultrasound transducers can acquire the ultrasound data. Moreover, the ablation electrode 20 comprises irrigation openings 28, 29 for allowing irrigation fluid flowing within the ablation catheter 4 to leave the tip 5 of the ablation catheter 4.

The ablation electrode 20 is electrically connected with an ablation control unit 7 by using an electrical conductor like a wire (not shown in FIG. 2 for clarity reasons), in order to allow a physician to control the application of ablation energy. In this embodiment the ablation control unit 7 comprises a radio frequency source for applying radio frequency energy to cardiac tissue for ablating the same. The ablation apparatus further comprises an irrigation control unit 8 for controlling the flow of the irrigation fluid within the ablation catheter 4 and, thus, for controlling the irrigation fluid leaving the tip 5 of the ablation catheter 4 through the irrigation openings 28, 29. The ablation catheter 4 comprises a lumen for guiding the fluid from the irrigation control unit 8 to the irrigation openings 28, 29. The irrigation control unit 8 preferentially comprises a fluid source and a pump for providing fluid to the tip 5 of the ablation catheter 4.

The ablation apparatus 1 further comprises an ultrasound control unit 9, which is electrically connected to the ultrasound transducers in the tip 5 of the ablation catheter 4 via electrical connections like electrical wires (not shown in FIG. 2 for clarity reasons). The ultrasound control unit 9 and the ultrasound transducers in the tip 5 of the ablation catheter 4 are preferentially configured such that the ultrasound data are acquired in the following way.

In a transmit mode a pulsed signal is generated, for instance, by the ultrasound control unit 9, which causes the respective transducer, in particular, the respective piezoelectric transducer, to transmit an ultrasound wave into the cardiac tissue. Then, the ultrasound data acquisition system formed by the ultrasound control unit 9 and the respective ultrasound transducer is switched from the transmit mode to a receive mode. In the receive mode ultrasound waves from the cardiac tissue are received by the respective ultrasound transducer, wherein the respective ultrasound transducer generates an electrical signal, which is preferentially amplified, converted to the digital domain and optionally pre-filtered to reduce noise. The resulting set of acquired data samples belonging to a single transmit pulse is called an RF-line. FIG. 3 shows exemplarily such an RF-line 60, wherein the amplitude A in arbitrary units is shown depending on the time t in arbitrary units. The ultrasound control unit 9 can be adapted to apply an envelope detection algorithm to the respective RF-line for creating an A-line 61, which is also exemplarily shown in FIG. 3.

The time axis in FIG. 3 can be related to different depths within the cardiac tissue such that each RF-line or A-line can be regarded as providing the amplitude depending on the depth within the cardiac tissue. Each transducer acquires several RF-lines over time such that each transducer acquires temporally dependent ultrasound data providing an amplitude value depending on the depth within the cardiac tissue and the time. In particular, for each transducer an M-mode image is acquired in the respective acquisition direction.

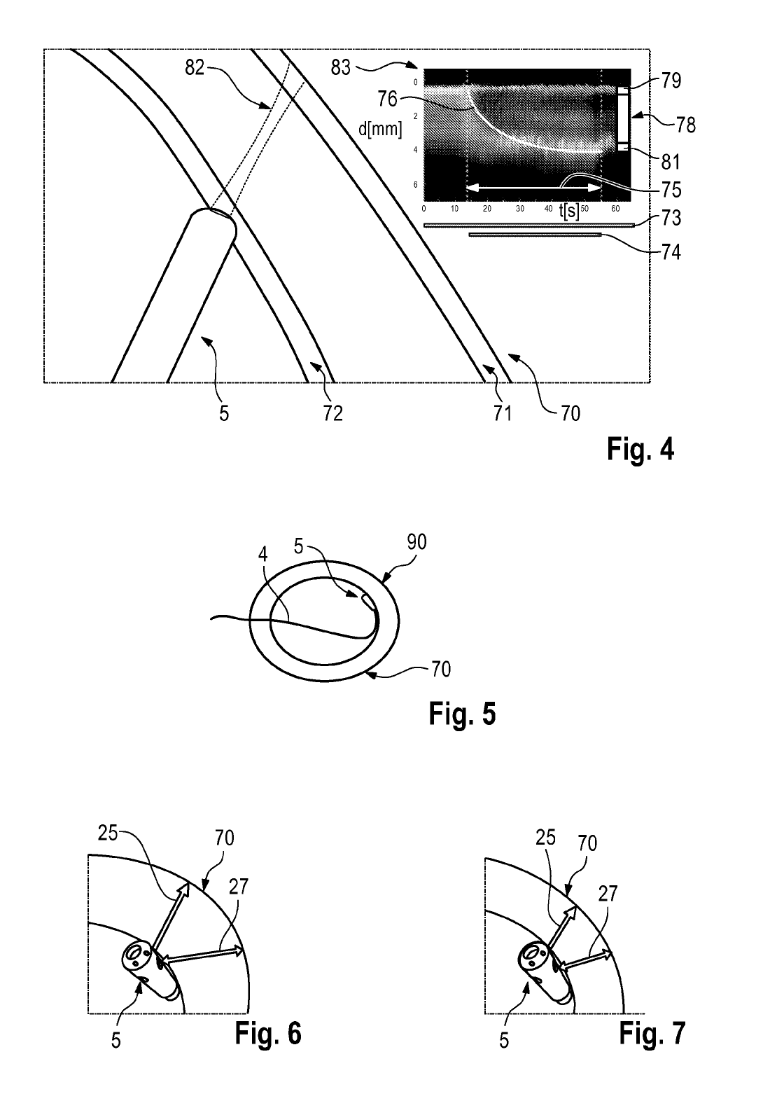

The ablation apparatus 1 is used for performing and for monitoring the cardiac ablation procedure. The ablation apparatus 1 is particularly adapted to cure cardiac arrhythmia. The ultrasound transducers in the tip 5 of the ablation catheter 4 enable a physician in an electrophysiology laboratory to assess in realtime certain relevant parameters of the heart wall from the inside. This will in the following exemplarily be illustrated with reference to FIG. 4.

FIG. 4 shows the tip 5 of the ablation catheter 4 acquiring ultrasound data by using the axial frontal transducer in the first acquisition direction. A corresponding ultrasound beam is schematically indicated in FIG. 4 by broken lines 82. The ultrasound waves are sent into a heart wall 70 and scattered and/or reflected ultrasound waves are received by the frontal axial transducer at the tip 5 of the ablation catheter 4. Resulting temporally dependent ultrasound data, i.e. in this embodiment a resulting M-mode image 83, is exemplarily shown in the upper right part of FIG. 4. The M-mode image 83 shows the ultrasound signal amplitude depending on the depth d in millimeters and depending on the time t in s. The line 73 indicates the duration of ultrasound monitoring and the lines 74, 75 indicate the duration of applying ablation energy to the cardiac tissue. The line 76 indicates the ablation depth and the column 78 indicates the position of a front side 72 of the heart wall 70 by using the block 79 and the ablation depth by using the block 81. By visual inspection of the ultrasound M-mode image 83 the physician can measure the heart wall thickness, i.e. the positions of the front side 72 and the back side 71 of the heart wall 70, and can then decide on the best ablation regime like the optimal ablation power, the optimal flow rate of the irrigation fluid being preferentially a saline cooling fluid and the optimal ablation duration. During the application of the ablation energy the lesion formation can be monitored, wherein the physician can halt the ablation procedure, when a lesion has become transmural, i.e. when the treatment reached the back side 71 of the heart wall 70. In case steam pockets are formed inside the cardiac tissue, the physician can see this formation in the M-mode image 83 and can halt the ablation procedure to prevent a tissue rupture, i.e. to prevent a so-called “pop”.

FIG. 4 shows schematically and exemplarily a certain orientation of the tip 5 of the ablation catheter with respect to the heart wall 70. The tip 5 of the ablation catheter can of course also be oriented in another way with respect to the heart wall 70. For instance, as schematically and exemplarily shown in FIG. 5, also the lateral transducers can be directed towards the heart wall 70.

FIG. 5 shows the ablation catheter 4 introduced into the left atrium 90 of the heart, wherein FIG. 6 shows the orientation of the tip 5 of the ablation catheter 4 in more detail in the systole and FIG. 7 shows the orientation of the tip 5 of the ablation catheter 4 in more detail in the diastole. It can be seen in FIGS. 6 and 7 that a certain number of transducers look to the outside of the heart chamber and the others look to the inside of the heart chamber.

Generally, an interpretation of the acquired ultrasound data, for instance, of the ultrasound M-mode image 83 shown in FIG. 4, is difficult, if the orientation of the tip 5 of the ablation catheter 4 with respect to the cardiac tissue, which may change in a moving environment such as a beating heart where the intrinsic motion of the heart modulates on the breathing motion of the lungs, is not known. The ablation apparatus 1 is therefore adapted to determine the spatial configuration in the surrounding of the ultrasound device 40, i.e. in the surrounding of the tip 5 of the ablation catheter 4, based on the acquired ultrasound data. In this embodiment the ablation apparatus 1 is adapted to determine the orientation of the ultrasound device 40 and the heart wall 70 in the surrounding of the ultrasound device 40 with respect to each other as a spatial configuration. In other words, in this embodiment the ablation apparatus 1 is adapted to determine the orientation of the ultrasound device 40 and, thus, of the tip 5 of the ablation catheter 4 relative to the heart wall 70 as the spatial configuration. The ultrasound transducers at the tip 5 of the ablation catheter 4 are therefore not only used for realtime monitoring of lesion progression as described above with reference to FIG. 4, but also for determining the spatial configuration in the surrounding of the ultrasound device, in particular, the orientation and optionally also the position of the ultrasound device and, thus, of the tip 5 of the ablation catheter 4 with respect to the cardiac tissue, without necessarily requiring an incorporation of additional sensors.

For determining the spatial configuration in the surrounding of the ultrasound device, in particular, the orientation of the tip 5 of the ablation catheter 4, the ablation apparatus 1 further comprises an ultrasound data processing unit 11 for processing the acquired first and second ultrasound data for determining motion data and/or distance data in the surrounding of the ultrasound device 40 in the first to fourth acquisition directions 24 . . . 27. The ablation apparatus 1 also comprises a spatial configuration determination unit 12 for determining the spatial configuration in the surrounding of the ultrasound device 40 based on the determined motion data and/or the determined distance data in the different acquisition directions 24 . . . 27. The ultrasound data processing unit 11 and the spatial configuration determination unit 12 form a spatial configuration determination apparatus for determining a spatial configuration in the surrounding of the ultrasound device based on the acquired ultrasound data. In this embodiment the ultrasound data processing unit 11 is adapted to determine motion data and distance data to be used by the spatial configuration determination unit 12 for determining the spatial configuration in the surrounding of the ultrasound device. In particular, in this embodiment the spatial configuration determination unit 12 is adapted to determine the orientation of the ultrasound device 40 and, thus, of the tip 5 of the ablation catheter 4 with respect to the heart wall as the spatial configuration.

The ultrasound data processing unit 11 is preferentially adapted to determine motion data being indicative of the magnitude and/or the direction of a motion between the ultrasound device and an object in the surrounding of the ultrasound device being, in this embodiment, the heart wall in the different acquisition directions. Moreover, the ultrasound data processing unit 11 is adapted to determine for each acquisition direction distance data being indicative of a distance between the ultrasound device 40 and the object in the surrounding of the ultrasound device 40 from the ultrasound data acquired in the respective acquisition direction.

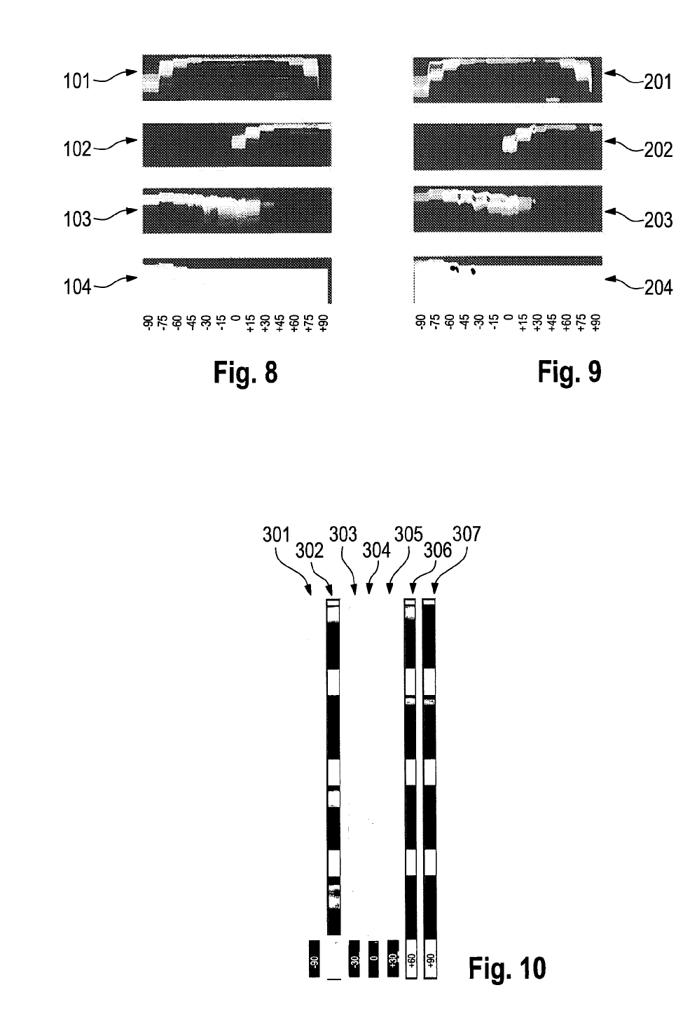

FIG. 8 shows ultrasound data, which have been acquired in the different acquisition directions, while the orientation of the ultrasound device and, thus, of the tip of the ablation catheter relative to the heart wall was known. In FIG. 8 the vertical axes indicate the distance to the respective ultrasound transducer and the horizontal axes indicate the orientation of the tip of the ablation catheter relative to the heart wall. In FIG. 8 an angle of zero degrees corresponds to a perpendicular orientation, in which the tip of the ablation catheter is perpendicular to the heart wall, and angles of +90 degrees and −90 degrees corresponds to parallel orientations, in which the tip of the ablation catheter is parallel to the heart wall. In FIG. 8 A-lines are shown, wherein for each angle several A-lines are measured over time, wherein the first ultrasound data 101 have been acquired in the first acquisition direction 26, the second ultrasound data 102 have been acquired in the second acquisition direction 24, the third ultrasound data 103 have been acquired in the third acquisition direction 27 and the fourth ultrasound data 104 have been acquired in the fourth acquisition direction 25. To these ultrasound data 101 . . . 104 a motion analysis can be applied for determining motion data.

The motion analysis is preferentially performed in each acquisition direction on a set of subsequent A-lines acquired in the respective angular orientation. During the acquisition of the A-lines they may be collected in a memory, wherein, if a new A-line comes in, the oldest A-line in memory may be removed and the new A-line may be appended in the memory. The motion estimation may be performed at each time t when a new A-line has been acquired and stored in the memory.

A set of subsequent A-lines at a respective angular orientation can be regarded as being a two-dimensional image to which techniques of motion estimation known from the field of image processing can be applied. These techniques require two images, which represent two different times, and produce a displacement vector or so-called motion vector (vₓ, v_{y}) for each location in the image. This vector describes the displacement of a pixel between the two images measured in amount of horizontal pixels (vₓ) and vertical pixels (v_{y}). For determining the motion data for a certain angular orientation the first image, which may be regarded as being a first sub-M-mode image, can be defined by the A-lines from time t-t₀ until the time t, wherein the time t may be regarded as being the current time, and the second image, which may be regarded as being a second sub-M-mode image, can be defined by the A-lines from the time t-t_{d}−t₀ until the time t-t_{d}. The value of t_{d} is preferentially relatively small, for example, corresponding to only a single or few A-lines.

For determining the motion data only the vertical component v_{y} of the motion vector is preferentially used. It is possible to only use the absolute value of the vertical component of the motion vector, which may be called mobility value, or to use directly the vertical component of the motion vector, in order to consider also the direction of the motion, i.e. to consider the magnitude and the direction of the motion.

Known motion estimation techniques, i.e. known motion determination algorithms, can be applied to the first and second images. For instance, known correlation-based techniques or known optical flow techniques can be used as disclosed in the articles “An iterative image registration technique with an application to stereo vision” by B. Lucas et al, Proceedings of Imaging Understanding Workshop, pages 121 to 130 (1981) and “Generalized Image Matching by the Method of Differences”, PhD thesis by B. Lucas, Carnegie-Mellon University, Department of Computer Science (1984), which are herewith incorporated by reference.

FIG. 9 shows exemplarily resulting mobility values, wherein first mobility values 201 have been determined based on the first ultrasound data 101, second mobility values 202 have been determined based on the second ultrasound data 102, third mobility values 203 have been determined based on the third ultrasound data 103 and fourth mobility values 204 have been determined based on the fourth ultrasound data 104. As can be seen in FIG. 9, the orientation angle, which may also be regarded as being a contact angle, of the tip of the ablation catheter has a certain mobility signature across the four transducers, which is different for the different orientation angles. The differences between the mobility signatures for different orientation angles are even more visible in FIG. 10, which shows seven motion signatures 301 . . . 307 corresponding to the orientation angles −90 degrees, −70 degrees, −30 degrees, 0 degrees, +30 degrees, +60 degrees and +90 degrees, respectively.

As can be seen in FIG. 8, from the ultrasound data transducer-to-tissue distances can be determined, which are different for the different orientation angles. The ultrasound data processing unit 11 is therefore adapted to determine the distance data from the ultrasound data in the different acquisition directions. For instance, a thresholding can be applied to the ultrasound data shown in FIG. 8 for estimating a distance between a respective transducer and the heart wall, in particular, the cardiac tissue. Starting from the beginning of an A-line the signal strength can be compared to a threshold, wherein the position, at which the signal strength exceeds the threshold, can be used to define the distance between the respective transducer and the heart wall in the respective acquisition direction and in the respective angular orientation. The threshold can be predefined based on experiments with known positions of the heart wall and/or the threshold can be determined based on a known or estimated noise level of the ultrasound measurement, wherein, if the signal strength exceeds the noise level, it may be concluded, that the heart wall is present. For determining the distance of the heart wall to the respective transducer a respective single A-line can be used or several consecutive A-lines can be averaged and the resulting average value can be compared with the threshold for determining the distance between the respective ultrasound transducer and the heart wall.

For determining the actual orientation angle the spatial configuration determination unit 12 uses a statistical classifier, wherein the statistical classifier is adapted to determine which orientation angle from a set of predefined orientation angles corresponds most likely to the determined motion and distance data derived from the actual ultrasound data, wherein the most likely orientation angle from the set of predefined orientation angles is determined as the actual orientation angle. For instance, in this embodiment the predefined orientation angles can be orientation angles from −90 degrees to +90 degrees with an increment of 15 degrees. The statistical classifier can be adapted such that it determines the orientation angle from this set of predefined orientation angles, which corresponds most likely to the motion and distance data derived from the actually measured ultrasound data.

The statistical classifier can be, for instance, a neural network such as a multi-layer perceptron as disclosed, for instance, in the article “Multilayer Perceptron, Fuzzy Sets, and Classification” by S. Pal and S. Mitra, IEEE Transactions on Neural Networks, volume 3, number 5, pages 683 to 697 (1992), which is herewith incorporated by reference. Also any other classifier scheme may be used for determining the actual orientation angle based on the motion and distance data derived from the actually measured ultrasound data.

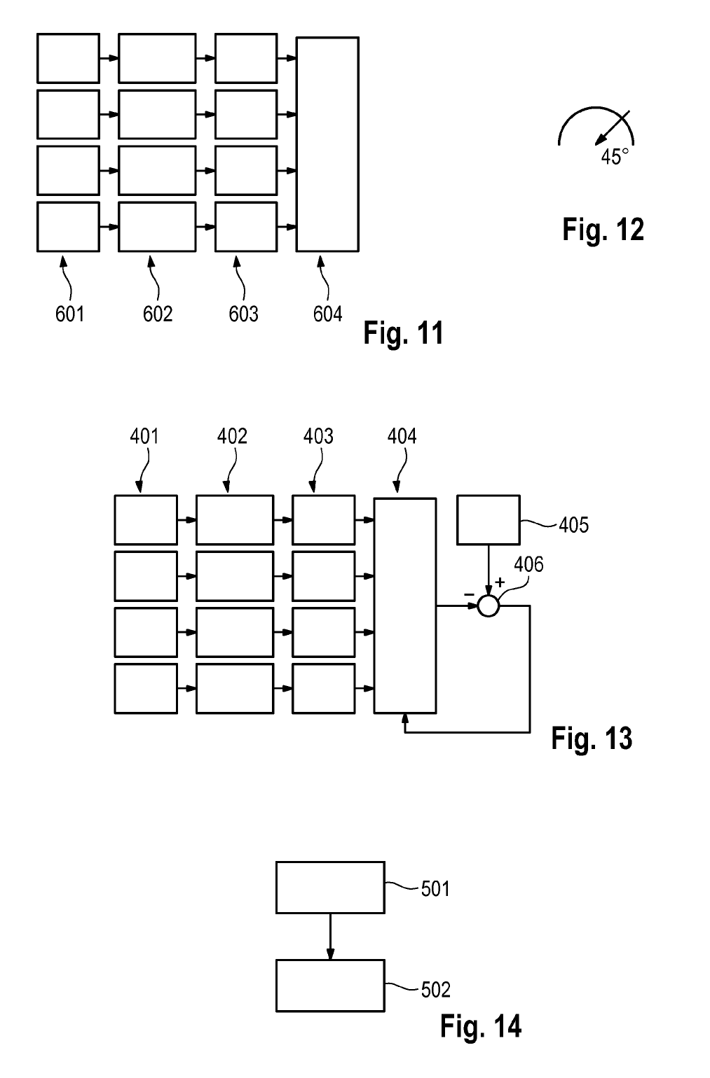

FIG. 11 illustrates the general scheme of the preferred orientation angle estimation procedure. The boxes 301 represent the acquisition of the first to fourth ultrasound data in the respective different acquisition directions. The ultrasound data are A-lines, which are collected in a memory. The collection of the A-lines in the memory is symbolized by the boxes 302. Then, the A-lines are used to determine the motion and the distance, i.e. the distance between the heart wall and the respective ultrasound transducer, in the respective acquisition direction. This determination of the motions and the distance based on the collected A-lines is represented by the boxes 303 in FIG. 11. The determined motions and distance are input into a classification logic represented in FIG. 11 by the box 304. The classification logic is the statistical classifier, which estimates the orientation angle depending on the determined motions and distance. This procedure may be performed in realtime such that a physician can see at every moment how the tip of the ablation catheter is oriented with respect to the heart wall inside the heart. The determined orientation angle can be visualized on a display 13 of the ablation apparatus 1, for instance, as shown in FIG. 12.

The statistical classifier has been trained by using training ultrasound data, wherein it is known to which angular orientation the training ultrasound data correspond. From the training ultrasound data motion data and distance data are determined in the different acquisition directions, wherein the statistical classifier is trained such that given the determined motion and distance data the known respective angular orientation is the most likely one. These training ultrasound data can be determined, for instance, by a benchtop study, wherein artificially moving tissue is mounted in a setup which allows fixing a tip of a catheter with the ultrasound device relative to the tissue in a desired known orientation angle. It is also possible to measure the orientation and/or position of the ultrasound device relative to the tissue in vivo by another means, i.e. not by the ultrasound device, while the training ultrasound data are acquired. The training of the statistical classifier will in the following exemplarily be described with reference to FIG. 13.

For a known orientation angle training ultrasound data are acquired in the different acquisition directions as indicated by the boxes 401. The training ultrasound data are formed by A-lines, which are collected in a memory as indicated by the boxes 402. The collected A-lines are then used for determining the motions and the distances in the respective acquisition directions. This is represented by the boxes 403. The determined motions and distances are input into the statistical classifier 404 to be trained, wherein the statistical classifier 404 provides an initial estimated orientation angle at the beginning of the training process. Then, as indicated by the circle 406, the estimated orientation angle is compared with the known real orientation angle 405, which in the present case forms the ground truth data. The comparison result, which can be regarded as being an estimation error, is fed back to the statistical classifier 404, wherein the statistical classifier 404 is modified, in order to reduce the estimation error. For instance, coefficients of the statistical classifier can be updated. Then, the statistical classifier 404 estimates the orientation angle again, wherein the newly estimated orientation angle is compared with the known real orientation angle for generating an updated estimation error in step 406. The steps of modifying the statistical classifier, estimating an orientation angle, comparing the estimated orientation angle with the known real orientation angle for generating an estimation error and feeding back the estimation error to the statistical classifier are iteratively performed such that the estimation error is minimized. After the estimation error has been minimized, the training of the statistical classifier has been completed, for instance, coefficients of the statistical classifier have been determined and are now frozen, wherein the trained statistical classifier can be used for determining an orientation angle based on actual ultrasound data as described above with reference to, for instance, FIG. 11.

Although in above described embodiments the motion data are mobility values, i.e. absolute values of the vertical components of the motion vectors, in other embodiments the motion derived from the actual ultrasound data can include directly the vertical component of the motion vector, i.e. also the direction of the motion in the respective acquisition direction can be considered. This can further improve the quality of the determination of the orientation angle, because the heart chamber is smaller in size during the contraction phase and larger in size during the rest phase as illustrated in FIGS. 6 and 7. Transducers that point outwards to the heart tissue will therefore measure heart contraction motion in synchrony with the heart activity, whereas transducers looking away from the heart tissue will not measure this motion.

The ablation apparatus and, thus, the spatial configuration determination apparatus cannot only be adapted to determine the orientation angle, but they can also be adapted to determine another kind of spatial configuration in the surrounding of the ultrasound device, in particular, in the surrounding of the tip of the ablation catheter. Generally, the statistical classifier can be adapted to determine which spatial configuration from a set of predetermined spatial configurations corresponds most likely to the acquired ultrasound data, wherein the most likely spatial configuration from the set of predefined spatial configurations is determined as the spatial configuration. The set of predefined spatial configurations can include, for instance, a spatial configuration, in which the ultrasound device is buried in an object, a spatial configuration, in which the ultrasound device is located within the apex of the heart, or a spatial configuration, in which the ultrasound device is located in a trabeculated structure of the heart. Thus, the ablation apparatus and the spatial configuration determination apparatus can be adapted to enable the recognition of special cases like a case, in which a tip of an ablation catheter is buried into cardiac tissue or a case in which the tip of the ablation catheter reaches into the apex or into a trabeculated structure of the heart. Also these situations have special motion data and distance data combinations in the different acquisition directions.

The ablation apparatus further comprises a navigation unit 10 for allowing the ablation catheter 4, in particular, the tip 5 of the ablation catheter 4, to be navigated to a desired location within the person 2. The navigation unit 10 can be adapted to allow a user to navigate the ablation catheter 4 completely by hand or semi-automatically. The ablation catheter 4 comprises built-in guiding means (not shown in FIG. 1), which can be controlled by the navigation unit 10. The ablation catheter 4 can, for example, be steered and navigated by using steering wires, in order to guide the tip 5 of the ablation catheter to a desired location within the person 2.

In the following an embodiment of a spatial configuration determination method for determining a spatial configuration in the surrounding of an ultrasound device will exemplarily be described with reference to a flowchart shown in FIG. 14.

The spatial configuration determination method is adapted to determine the spatial configuration based on acquired ultrasound data, which have been acquired by the ultrasound device in different acquisition directions. In step 501 the ultrasound data acquired for the different acquisition directions are processed for determining in each acquisition direction motion data and/or distance data, wherein the motion data represent motion of an object in the surrounding of the ultrasound device in the respective acquisition direction and wherein the distance data represent a distance between the object and the ultrasound device in the respective acquisition direction. In particular, the distance of the respective ultrasound transducer to the cardiac tissue and the motion of the cardiac tissue relative to the respective ultrasound transducer are determined in the respective acquisition direction for determining distance data and motion data in the respective acquisition direction. This determination is performed for all acquisition directions, in order to determine the motion data and the distance data in all acquisition directions. In step 502 the spatial configuration in the surrounding of the ultrasound device, in particular, the orientation angle between the ultrasound device and, thus, a catheter tip at which the ultrasound device may be attached and a heart wall may be determined based on the motion data and/or distance data, which have been determined in the different acquisition directions, by a spatial configuration determination unit. Preferentially, a statistical classifier is used for determining the spatial configuration based on the motion and distance data, which have been determined for the different acquisition directions.

The ablation apparatus, in particular, the spatial configuration determination apparatus, is preferentially adapted to estimate and visualize the current catheter tip orientation with respect to the inner heart wall, wherein the estimation is based solely on the ultrasound data acquired from the same catheter tip. The current practice in electrophysiology is to treat arrhythmia with radio frequency ablation catheters, wherein the position of the ablation catheter is monitored by fluoroscopy. This technique has the disadvantage that soft tissue does not provide contrast in the fluoroscopic images which makes catheter orientation estimation by fluoroscopy not possible. Therefore, for determining the catheter orientation and the catheter position within the heart the catheter tip is also often tracked by using an electromagnetic technique, whereby a sensor is placed in the tip of the catheter, which senses varying magnetic fields generated by an external field generator, wherein the orientation and the position of the ablation catheter is calculated depending on the sensed varying magnetic fields. This electromagnetic technique has the disadvantage that it needs the integration of three-dimensional sensors in the already restricted space inside the ablation catheter and that an additional external magnetic field generator has to be placed in the laboratory. Moreover, this electromagnetic technology cannot fulfill the ultimate clinical need of following the ablation front in the tissue during the arrhythmia treatment.

In the embodiment described above with reference to FIG. 2 the ultrasound device at the tip of the ablation catheter comprises three lateral ultrasound transducers equidistantly arranged, i.e. with a respective angular distance of 120 degrees, and a frontal ultrasound transducer. The number of ultrasound transducers, for instance, the number of lateral transducers around the circumference of the tip of the ablation catheter or the number of ultrasound transducers in the axial direction can be increased, in order to refine and increase the accuracy of determining the spatial configuration in the surrounding of the ultrasound device. An equidistant arrangement of the lateral transducers on the circumference of the ablation catheter is preferred, because in this case an axial rotation of the ablation catheter does not have a major influence on the accuracy of the determination of the orientation angle.

The determination of the orientation of the tip of the ablation catheter and optionally also of the position of the tip of the ablation catheter is preferentially based on the fact that different sets of observed tissue motions towards and away from the transducer are achieved for different catheter orientations and different sets of observed distances between the transducers and the cardiac tissue are achieved for different catheter positions. For these reasons, for each ultrasound transducer signal a motion analysis is preferentially performed, which reveals changes due to motion between incoming ultrasound datA-lines over time. The combination of the motion information coming from the different ultrasound transducers is indicative of the orientation of the tip of the ablation catheter with respect to the cardiac tissue such that this combination of motion information can be used optionally together with the distance information for determining the orientation and optionally the position of the tip of the ablation catheter. The determined position of the tip of the ablation catheter is the position with respect to the surrounding cardiac tissue.

Although in above described embodiments the introduction apparatus is an ablation apparatus, wherein the introduction element is an ablation catheter, in other embodiments the introduction apparatus can also be another apparatus for introducing an introduction element into an object. For instance, the introduction apparatus can be adapted to introduce another kind of catheter or another interventional instrument like a needle into the object, wherein also this interventional instrument can be equipped with an ultrasound device for acquiring ultrasound data in different acquisition directions, which can be used for determining the spatial configuration in the surrounding of the ultrasound device. Correspondingly, the introduction apparatus, in particular, the spatial configuration determination apparatus, can be used in other interventional procedures not being a cardiac ablation procedure.

Although in above described embodiments motion data and distance data are determined based on the acquired ultrasound data, wherein the motion data and the distance data are used for determining the spatial configuration in the surrounding of the ultrasound device, in other embodiments also only motion data or only distance data can be used for determining the spatial configuration.

Although in above described embodiments A-lines are used for determining motion and position data, in other embodiments also another kind of temporal ultrasound data can be used for determining, for instance, motion and distance data. For example, RF-lines can directly be used, without determining A-lines, for determining the distance data and the motion data.

Although in an above described embodiment the determined orientation angle is shown on a display in accordance with FIG. 12, in another embodiment the orientation angle and optionally also another aspect of the spatial configuration can be shown in another way. For instance, a realtime computer animation of the tip of the catheter and the spatial configuration in the surrounding of the tip can be shown, for instance, similar to FIGS. 6 and 7. The computer animation may also show, for example, if tissue is folded around the tip.

Other variations to the disclosed embodiments can be understood and effected by those skilled in the art in practicing the claimed invention, from a study of the drawings, the disclosure, and the appended claims.

In the claims, the word “comprising” does not exclude other elements or steps, and the indefinite article “a” or “an” does not exclude a plurality.

A single unit or device may fulfill the functions of several items recited in the claims. The mere fact that certain measures are recited in mutually different dependent claims does not indicate that a combination of these measures cannot be used to advantage.

Procedures like the determination of the A-lines based on the RF-lines, the determination of motion data, the determination of distance data, the determination of the spatial configuration in the surrounding of the ultrasound device, et cetera performed by one or several units or devices can be performed by any other number of units or devices. These procedures and/or the control of the spatial configuration determination apparatus in accordance with the spatial configuration determination method can be implemented as program code means of a computer program and/or as dedicated hardware.

A computer program may be stored/distributed on a suitable medium, such as an optical storage medium or a solid-state medium, supplied together with or as part of other hardware, but may also be distributed in other forms, such as via the Internet or other wired or wireless telecommunication systems.

Any reference signs in the claims should not be construed as limiting the scope.

The invention relates to a spatial configuration determination apparatus for determining a spatial configuration in the surrounding of an ultrasound device, in particular, for determining the orientation of the ultrasound device and of an object in the surrounding of the ultrasound device with respect to each other. The ultrasound device is preferentially arranged at a tip of a cardiac ablation catheter such that, for instance, the orientation of the tip relative to cardiac tissue can be determined. Motion data and/or distance data are determined in different directions from ultrasound data acquired by the ultrasound device, wherein the determined motion data and/or distance data are used for determining the spatial configuration. This allows determining, for instance, the orientation of a tip of an ablation catheter relative to cardiac tissue, without necessarily requiring further orientation determination means.

1. An introduction apparatus for introducing an introduction element into an object, the introduction apparatus comprising:
the introduction element to be introduced into the object, wherein the introduction element comprises single ablation means at a tip of the introduction element,
an ultrasound device for acquiring first and second ultrasound data in different acquisition directions, wherein the ultrasound device is arranged at the introduction element,
a spatial configuration determination apparatus for determining the spatial configuration around the ultrasound device within the object, the spatial configuration determination apparatus being adapted to determine the spatial configuration based on the first and second ultrasound data, wherein the spatial configuration determination apparatus comprises:
an ultrasound data processing unit for processing the acquired first and second ultrasound data for determining motion data representing motion of an object in the surrounding of the ultrasound device and/or distance data representing a distance between the object and the ultrasound device in the first and second acquisition directions,
a spatial configuration determination unit for determining the spatial configuration in the surrounding of the ultrasound device based on the motion data and/or the distance data determined for the different acquisition directions.

2. The introduction apparatus as defined in claim 1, wherein the spatial configuration determination unit is adapted to determine the orientation and/or the position of the ultrasound device and the object in the surrounding of the ultrasound device with respect to each other as the spatial configuration.

3. The introduction apparatus as defined in claim 1, wherein the ultrasound data processing unit is adapted to determine motion data being indicative of the magnitudes and/or the directions of the motion between the ultrasound device and the object in the surrounding of the ultrasound device in the first acquisition direction from the first ultrasound data and of the motion between the ultrasound device and the object in the surrounding of the ultrasound device in the second acquisition direction from the second ultrasound data.

4. The introduction apparatus as defined in claim 3, wherein the acquired first ultrasound data form a first M-Mode image and the acquired second ultrasound data form a second M-mode image, wherein the ultrasound data processing unit is adapted to determine first sub-M-mode images from the first M-mode image and second sub-M-mode images from the second M-mode image and to apply a motion determination algorithm to the first sub-M-mode images for determining first motion data being indicative of the motion in the first acquisition direction and to apply the motion determination algorithm to the second sub-M-mode images for determining second motion data being indicative of the motion in the second acquisition direction.

5. The introduction apparatus as defined in claim 1, wherein the ultrasound data processing unit is adapted to determine the distance data by thresholding the first and second ultrasound data.

6. The introduction apparatus as defined in claim 1, wherein the spatial configuration determination unit is adapted to use a statistical classifier for determining the spatial configuration in the surrounding of the ultrasound device based on the motion data and/or the distance data in the different acquisition directions.

7. The introduction apparatus as defined in claim 6, wherein the statistical classifier is adapted to determine which spatial configuration from a set of predefined spatial configurations corresponds most likely to the motion data and/or the distance data in the different acquisition directions, wherein the most likely spatial configuration from the set of predefined spatial configurations is determined as the spatial configuration.

8. The introduction apparatus as defined in claim 7, wherein the set of predefined spatial configurations includes at least one of the group of a spatial configuration, in which the ultrasound device is buried in an object; a spatial configuration, in which the ultrasound device is located within the apex of a heart; a spatial configuration, in which the ultrasound device located in a trabeculated structure of the heart; predefined orientations of the ultrasound device and an object in the surrounding of the ultrasound device with respect to each other; predefined positions of the ultrasound device and an object in the surrounding of the ultrasound device with respect to each other.

9. The introduction apparatus as defined in claim 1, wherein the ultrasound data are provided as RF-lines or A-lines.

10. (canceled)

11. The introduction apparatus as defined in claim 1, wherein the ultrasound device is arranged at a tip of the introduction element and comprises a frontal transducer for acquiring the first ultrasound data in a frontal direction with respect to the tip of the introduction element and at least one lateral transducer for acquiring the second ultrasound data in a lateral direction with respect to the introduction element.

12. The introduction apparatus as defined in claim 11, wherein the ultrasound device comprises at least three lateral transducers for acquiring the second ultrasound data and for acquiring third and fourth ultrasound data, wherein the ultrasound device is adapted such that the first to fourth ultrasound data are all acquired in different acquisition directions, wherein the spatial configuration determination apparatus is adapted to determine the spatial configuration around the ultrasound device based on the first to fourth ultrasound data.

13. (canceled)

14. A spatial configuration determination computer program for determining a spatial configuration in the surrounding of an ultrasound device, the computer program comprising program code means for causing an introduction apparatus as defined in claim 1 to carry out the steps of a spatial configuration determination method for determining a spatial configuration in the surrounding of an ultrasound device, when the computer program is run on a computer controlling the spatial configuration determination apparatus, the spatial configuration determination method being adapted to determine the spatial configuration based on acquired first and second ultrasound data, which have been acquired by the ultrasound device in first and second acquisition directions, respectively, wherein the first and second acquisition directions are different and wherein the spatial configuration determination method comprises:
processing the acquired first and second ultrasound data for determining motion data representing motion of an object in the surrounding of the ultrasound device and/or distance data representing a distance between the object and the ultrasound device in the first and second acquisition directions by an ultrasound data processing unit,
determining the spatial configuration in the surrounding of the ultrasound device based on the motion data and/or the distance data, which have been determined in the different acquisition directions, by a spatial configuration determination unit.
